(19) United States
(12) Patent Application Publication
(10) Pub. No.: US 2005/0175688 A1
(43) Pub. Date: Aug. 11, 2005

(54) Method of treating pain in a subject by the administration of aminobenzoate potassium

(76) Inventor: Jaswant S. Chaddha, Villa Rica, GA (US)

Correspondence Address: Supervisor, Patent Prosecution Services;PIPER RUDNICK LLP, 1200 Nineteenth Street, N.W., Washington, DC, 20036-2412, US

(21) Appl. No.: 10/970,542

(22) Filed: Oct. 22, 2004

Related U.S. Application Data
(60) Provisional application No. 60/541,756, filed on Feb. 5, 2004.

(51) Int. Cl. A61K 31/195; A61K 9/20

(52) U.S. Cl. 424/464; 514/567

(57) ABSTRACT

Provided is a novel method of treating chronic pain symptoms associated with a fibrotic condition in a subject in need of such relief. Particularly provided is a method for treatment of chronic pain associated with fibrosis and resulting adhesions and, more particularly, a method for treating chronic pelvic pain in women, such as that experienced by women suffering from endometriosis.

This application claims priority from U.S. Provisional Application Ser. No. 60/541,756, filed Feb. 5, 2004. The entirety of that provisional application is incorporated herein by reference.

### BACKGROUND OF THE INVENTION

1. Field of the Invention

The present invention relates to a novel method of treating fibrotic conditions in a subject in need of such treatment. Particularly the present invention relates to a method of providing treatment to alleviate chronic pain symptoms associated with fibrotic conditions or fibrosis. More particularly, a method is provided for treating endometriosis in women and alleviating the chronic pelvic pain associated with endometriosis.

2. Background of the Technology

The human body responds to trauma and injury by scarring. Fibrotic conditions or Fibrosis, a type of disorder characterized by excessive scarring, occurs when the normal wound-healing response to trauma goes awry causing an excessive production and deposition of collagen. Fibrosis can result from diverse causes including trauma, surgery, infection, endometriosis and any other unusual conditions or assaults on the tissues of the body. Fibrotic conditions can include, but are not limited to: cirrhosis of the liver, atherosclerosis, Dermatomyositis, Progressive Systemic Sclerosis, pulmonary fibrosis, connective tissue diseases, adhesions and endometriosis. Although fibrotic disorders can be acute or chronic, the disorders share the common characteristics of excessive collagen accumulation with an associated loss of function, increased adhesions between normal body tissues and organs, and pain. When tissues are irritated by an abnormal condition they are prone to scar and become fibrotic, particularly if the damage is repeated. Regardless of etiology, fibrosis manifests itself in an overproduction of fibrotic tissue that causes destructive and painful adhesions in the body.

Adhesions are strands of scar tissue (fibrin bands). In a fibrotic condition, adhesions connect tissues or structures that are normally separate. Adhesions in the abdomen or pelvic area can lead to infertility, pelvic pain, small bowel obstruction, or in extreme cases, the need for repeated surgeries.

Without great success, drug therapy and surgery have been employed by physicians to try to alleviate the debilitating effects of chronic pelvic pain caused by fibrosis and associated adhesions commonly found in women suffering from chronic pelvic pain/endometriosis.

Drugs approved in the U.S. for endometriosis consist of danazol and gonadotropin-releasing hormone (GnRH) analogs. In addition oral contraceptives often have been prescribed for women who suffer from chronic pelvic pain/endometriosis. In Europe, other drugs are also available for use, namely progestogens such as gestrionine; however, these are not approved for use in the United States. None of the conventional drug therapies approved for use in the United States or abroad have proven fully successful in the effort to alleviate the condition of chronic pelvic pain/endometriosis.

Surgical techniques to alleviate chronic pain associated with fibrosis have also been employed with little lasting success. In fact, the incidence of adhesions following abdominal surgery is cumulative with multiple surgeries. Female gynecological surgeries result in a particularly high rate of adhesions. In one study, autopsy investigations indicated a 90% incidence of adhesions in patients with multiple surgeries, 70% incidence of adhesions in patients with a gynecologic surgery, and a 50% incidence of adhesions in patients with no surgical history. Adhesions may occur as the result of tissue damage other than that caused by surgical procedures, including, but not limited to: traumatic injury; inflammatory disease; arthritic conditions in the joints of the hands, elbows, shoulders, ankles, knees, hips, spine and neck due to the aging process or induced by autoimmune disease; intraperitoneal chemotherapy; radiation therapy; Interstitial Cystitis; and endometriosis.

The most frequent problem with adhesions is a constriction of the small intestine, producing constipation (sometimes complete bowel blockage, requiring emergency treatments). Abdominal pain, another common symptom of fibrosis and the associated adhesions, is caused when the bands of scar tissue bind up the internal organs so that movement pulls on them. Linkage of menstruation to changes in bowel function (e.g., inducing diarrhea) may occur as the result of scar tissue attaching the uterus to the intestine. Adhesions may also impair fertility in women by causing blockage of the fallopian tubes.

Although adhesions can be removed by surgical intervention (adhesiolysis), including laparoscopic techniques, recent studies suggest that the pain-relief benefit provided by such surgical procedures is often only short-term. Many patients are treated with multiple adhesiolysis procedures in an attempt to alleviate the painful symptoms of adhesions. Regrettably the result of repeated surgeries is often simply more extensive fibrosis and chronic pelvic pain. Each year over 400,000 adhesiolysis procedures are performed in the United States. Additionally, presacral neurectomy and utero-sacral nerve ablation have been used in attempts to alleviate the symptoms of chronic pelvic pain; all with very limited success.

Inadequate pharmacological treatments, inadequate and often aggravating repeated surgical procedures, and the little understood etiology of endometriosis with its accompanying fibrosis and painful adhesions has resulted in a growing chronic pelvic pain/endometriosis problem among reproductive-age women.

Endometriosis is one of the most significant diseases affecting young women. The prevalence of endometriosis is about 10 to 15 percent of women aged 15 to 45 years. In clinical practice, it is reasonable to first suspect endometriosis as the cause of pelvic pain in women of reproductive age; although other reasons for pelvic pain are known. Chronic pelvic pain that is associated with endometriosis is estimated to be 71%-87% of the population of women experiencing such pain. Endometriosis as the cause of chronic pelvic pain is laproscopically diagnosed 70%-80% of the time. Because of the high coincidence of endometriosis and chronic pelvic pain, even when a laproscopic examination seems negative, a diagnosis of endometriosis as the causal factor of chronic pelvic pain should still be considered in women who, after a thorough history and physical examination fail to reveal an alternative cause. Non-gynecologic etiology of chronic pelvic pain can include maladies that are gastrointestinal, urological, musculoskeletal, psychiatric, rheumatological, sexual abuse or physical abuse.

Beyond the personal toll in pain and suffering caused by chronic pelvic pain/endometriosis, the annual financial costs can be enormous. It is estimated that in the United States alone, there are 9.2 million women who suffer from chronic pelvic pain/endometriosis. The annual cost for medical management of this problem is estimated at $2.6 billion. In addition, the annual surgical management cost for chronic pelvic pain/endometriosis is estimated at $2.9 billion. Thus, beyond the personal burden endured by women suffering from chronic pelvic pain and/or endometriosis, the societal cost of this health problem is estimated to be at least $5.5 billion annually.

Although fibrosis, such as manifest in chronic pelvic pain/endometriosis, is widely prevalent, debilitating, and costly in both personal and economic losses, there is no effective treatment currently available. While the cause of endometriosis is uncertain, it is believed possibly to be an autoimmune disease. Endometriosis occurs when endometrial tissue or tissue resembling the lining of the uterus is found outside the uterus in the pelvic region. Through each menstrual cycle, this aberrant tissue will bleed cyclically. Such bleeding is intimately connected to menses and results eventually in fibrosis. Active and fibrotic endometrial tissue can result in adhesions within the pelvic area and cause severe pain during each menstrual cycle. Cycles of bleeding and fibrosis, especially in endometriosis nodules around the ovaries and fallopian tubes, can lead to infertility. Endometriosis can account for about 25 to 30 percent of unexplained infertility.

As earlier indicated, no oral medication is currently available to effectively treat chronic pelvic pain in patients clinically suspected as suffering from endometriosis. No one surgical treatment has been effective and often such well-intentioned surgical procedures serve only to aggravate the condition of chronic pelvic pain.

It remains therefore that the medical community is in need of an effective method of treating pain associated with fibrosis and adhesions and particularly for treating the chronic pelvic pain experienced by a very large number of reproductive-age women.

### SUMMARY OF THE INVENTION

The present invention is provided as a novel, effective, long-term method to treat adhesions attendant to a fibrotic condition, which can include any condition where excess collagen is being produced such as, for example, cirrhosis of the liver, atherosclerosis, Dermatomyositis, Progressive Systemic Sclerosis, pulmonary fibrosis, connective tissue diseases, adhesions and endometriosis.

Another embodiment of the present invention provides an effective method of treating abdominal and/or pelvic pain, the cause of which is fibrosis.

Another embodiment of the present invention provides an effective method of treating chronic pelvic pain associated with the condition of endometriosis.

Another embodiment of the present invention provides a method of effectively alleviating the symptom of pain normally associated with endometriosis.

Another embodiment of the present invention provides a method that includes a protocol of oral administration of aminobenzoate potassium for the treatment of pain caused by adhesions.

Another embodiment of the present invention provides a non-intrusive method for testing a subject suspected of suffering from chronic pelvic pain caused by endometriosis for the presence of that condition.

### DETAILED DESCRIPTION OF THE INVENTION

A novel method of treating fibrotic conditions has been discovered, which results from excess collagen production and subsequent adhesion formation. Particularly, a method of treating a subject suffering from chronic pain associated with fibrosis and the adhesions that result therefrom is provided.

As a non-limiting example, the following detailed description of the method of the present invention as applied to the treatment of chronic pelvic pain associated with endometriosis is provided. Endometriosis is typical of fibrotic conditions in general in that excess collagen production results in the formation of excess fibrous tissues, which bind to other tissues to form adhesions. As with other fibrotic conditions, movement of the adhesions and the bound tissues can cause chronic pain in the subject suffering from such a condition.

The method of the present invention includes a protocol of pharmacological treatment that requires the administering of aminobenzoate potassium (Potaba) to patients in need of relief from the symptom of pain associated with fibrosis and the adhesions that result from that fibrotic condition. Potaba is part of the Vitamin B complex. It is a naturally occurring compound that is known to participate in many biologically important processes.

While this theory of mechanism of action of the therapeutic effects of Potaba in patients is not limiting to the scope of the present invention, it is believed that Potaba has an antifibrotic effect due to increased oxygen uptake at the tissue level. For example, the fibrotic condition with the resultant adhesions is sequelae to endometriotic lesions as they heal. Endometriosis, believed to be an autoimmune disease, creates tissue lesions which the body responds to with the over-production of collagen and fibrous bands. These fibrous bands form adhesions between normal tissues, which subsequently cause chronic pain to the subject. It is believed that Potaba relieves the resulting chronic pelvic pain by an antifibrotic mechanism that releases oxygen to the fibrous tissue, which in turn promotes dissolving of the excess fibrous tissue.

Following a 48-month-long period, in which the Potaba protocol of the present invention was tested, it was discovered that administering relatively small doses of Potaba alone to volunteer women subjects of reproductive age, who were suffering from chronic pelvic pain/endometriosis, resulted in significantly effective pain relief. The total number of women under clinical examination was 212. The number of volunteer women subjects of reproductive age who participated in the inventive protocol of Potaba therapy to treat chronic pelvic pain/endometriosis was 25. The ages and number of subjects undergoing the test protocol of Potaba therapy is provided in Table 1.

TABLE 1

| AGE (YEARS) | NO. OF MONTHS IN THERAPY STUDY |
|---|---|
| 20-25 | 8 |
| 26-30 | 8 |
| 31-35 | 6 |
| 36-40 | 3 |

Preliminary to involvement in this voluntary study, the participating women patients were carefully screened to determine suitability for the study. Table 2 provides the parity (number of living children) data for the 25 women participating in the Potaba protocol study.

TABLE 2

| PARITY | NO. OF STUDY PARTICIPANTS |
|---|---|
| 0 | 5 |
| 1 | 4 |
| 2 | 9 |
| 3 | 6 |
| 4 | 1 |

Of the 25 women participating in the study of the Potaba protocol method of the present invention, five had undergone caesarean section procedures in the past.

Preliminary to commencing the Potaba protocol study, each of the 25 participants underwent an evaluation/a self test for endometriosis prepared by the Endometriosis Association. All of those who volunteered for the therapy protocol met the criteria of 10 points or more on the self-test, that threshold defining a subject who was at risk for endometriosis. Endometriosis was laparoscopically confirmed for 12 of the 25 participants. Importantly it should be noted that a negative laparoscopy examination for evidence of endometriosis does not rule out the possibility that endometriosis is the actual cause of the fibrosis and the resulting chronic pelvic pain. Due to the invasive nature of surgical techniques for diagnosis and the above recited fact that a negative surgical diagnosis does not necessarily rule out the presence of endometriosis, the early (pre-laproscopic) pharmacological treatment of suspected endometriosis has been widely accepted. The American College of Obstetrics and Gynecology Bulletin, 11, 1999 suggest the treatment of patients presenting chronic pelvic pain with GnRH analogues for at least 3 months or Danazol for at least 6 months prior to surgical evaluation or surgical treatment. Based on this fact, it was determined that Potaba should be administered to patients that are suspected of suffering from endometriosis when they present with chronic pelvic pain. This early treatment with Potaba can result in the expeditious relief of pain symptoms and, when so effective, can also serve to confirm the suspicion of an endometrious condition as a diagnosis.

A pelvic pain assessment which was originated by the International Pelvic Pain Society© 1999 (IPPS) was also conducted. IPPS evaluated the volunteer subjects for the following information: pain, menses, bladder, bowel habits, gastro-intestinal/eating habits, health habits, coping mechanisms, a short form McGill questionnaire (psychological; level of depression assay), daily activities, personal history, surgical history, sexual history and physical history. The results of this preliminary evaluation of the test subjects is provided in Table 3.

TABLE 3

| HISTORY AND PRESENTING | |
|---|---|
| SYMPTOMS | NO. OF STUDY PARTICIPANTS |
| Moderate Pelvic Pain | 8 |
| Severe Pelvic Pain | 17 |
| Dyspareunia | 20 |
| Dysmenorrhea | 25 |
| Backache | 14 |
| Bladder | 16 |
| Bowel | 16 |
| Gastrointestinal | 6 |
| Short-Form McGill Questionnaire | |
| Mild | 3 |
| Moderate | 9 |
| Severe | 14 |
| Other Areas of Pain | 14 |
| Sexual/Physical Abuse | 20 |

A thorough physical examination was also conducted for each of the 25 participants. The results of the pelvic examination are provided in Table 4.

TABLE 4

| PELVIC EXAMINATION | |
|---|---|
| FINDINGS | NO. OF STUDY PARTICIPANTS |
| Normal EGBUS/Vagina | 25 |
| Unilateral tenderness | 10 |
| Bilateral cervical motion | 15 |
| uterine, adnexal tenderness | |
| Per rectal findings confirmed | 15 |

Preliminary to being selected to participate in the Potaba protocol test, volunteer women underwent an initial clinic work-up to include: Physical examination and pelvic examination; lab test to include: CBC, complete metabolic panel including lipid panel, cervical cultures GC, Chlamydia, and ultrasound when deemed appropriate by the examining physician. Pelvic examinations and complete laboratory test were repeated every 8 weeks throughout the Potaba test period.

In addition to a careful review of the pre-test examinations of the prospective subjects, exclusion criteria were also established as a means of reducing unrelated influences into the test results. The established exclusion criteria for test subjects included: pregnant or pre-menopausal condition, not practicing birth control, presence of a chemically induced skin condition, lung or kidney disease, unusual muscle soreness or weakness, uncontrolled hypertension, hypoglycemia or diabetes, and allergy to Potaba.

The present invention includes the administration of aminobenzoate potassium (Potaba) with or without a suitable carrier to a subject in need of pain relief caused by a fibrotic condition. The Potaba can be administered to the subject by any means deemed appropriate by the attending physician although the preferred method is oral administration. While this method is preferred, it does not limit the invention from using other methods should it be, in special situations, ill-advised or impossible to orally administer the Potaba.

It is therefore fully within the concept of the present invention to administer Potaba to a patient by any suitable method to include, for example, oral (including delayed-release orally ingested capsules), rectal, transdermal (with or without use of a chemical and/or patch carrier), and direct peritoneal insertion (intraperiotoneal injection or infusion), a method that can also be accomplished through a laproscope, trocar, cannula or other similar avenue. It is also possible that the Potaba administration method of the present invention can be accomplished using a time-release pellet implant in the body tissues (such as in the buttocks or abdominal wall); vaginal insertion; inhalation delivery; contact lens delivery; IV, IM, or subcutaneous injections; and local direct application on adhesions. Such local applications can be accomplished using Potaba alone or in carriers such as lotions, creams, ointments or salves, which are applied directly to scars. It is also possible to apply creams or powders containing Potaba directly to tissues before or after excision of scar tissue. Any of the above listed delivery methods of Potaba can be modified or adapted so long as the application method of the present invention includes a therapeutic amount of Potaba.

An effective protocol of Potaba administration to a subject can be in daily doses of between about 2 grams and 30 grams of Potaba; and preferably between 5 grams and 20 grams of Potaba and most preferably between 10 and 15 grams of Potaba for an average adult subject. It is also within the scope of this invention to modify the dose levels disclosed herein to accommodate the possible use of this invention for pediatric or even veterinary applications. It is also within the concept of the invention to provide proportionately smaller doses multiple times throughout the course of a day. Further, it is within the scope of the present invention to administer the dose of Potaba to a subject less frequently than daily by using time-release vehicles, patch technology or any other avenue for the effective administration of the Potaba.

For purpose of the exemplary Potaba protocol study, each volunteer participant was administered Potaba in doses of 12 grams/day po (orally) either by capsules or envules to be dissolved in chilled water or juice. Only one physician conducted the physical evaluation of each of the participants to ensure consistency throughout the study. In addition to repeated laboratory and thorough physical examinations, the examining physician personally discussed and recorded responses to questions about the presence or absence of side-effects and improvements in symptoms, if any. The care exercised during the test period in providing consistent laboratory, physical and personal questioning in the repetitive evaluations of each volunteer participant served to reduce or eliminate the introduction of subjective data into the objective analysis of the effects of the Potaba protocol. Although the subjects individual responses to questions regarding symptoms and particularly levels of pain experience throughout the test period inherently can include a subjective element, the questions were repetitiously and precisely posed to the subjects so as to prompt a consistent response minimally effected by the questioner's choice of words, tone, delivery, etc. The test subjects reported pelvic pain experience as a number selected between the levels of 1 to 10, with 1 being no pain experienced and 10 being the most severe level of pain. Each participant's response was carefully recorded for each visit so as to provide a clear record of the level of pain progression that was easily correlated to the administration of Potaba and the progress of the Potaba protocol.

TABLE 5
Potaba Protocol Study Results

| Results | No. of Study Participants |
|---|---|
| Completed 48 week study period | 4 |
| Pain relief average (moderate) | 4 |
| Pain relief (complete) | 6 |
| Patients still in study protocol | 11 |
| Total Participants in Potaba Protocol | 25 |
| Discontinued Potaba protocol | |
| (reason) | |
| nausea | 3 |
| lost to follow-up | 5 |
| rash | 2 |

It was clearly demonstrated that a therapy that includes the administration of Potaba can significantly alleviate chronic pain in subjects suffering from the effects of adhesions caused by fibrosis, such as that found in endometriosis. As indicated in Table 5, six patients out of 25 patients who were participating in the Potaba protocol study obtained complete relief from pain. Four other patients participating in the Potaba protocol study obtained moderate relief from chronic pelvic pain. Ten of the 25 patients originally participating in the study discontinued the Potaba protocol for various reasons. Eleven patients out of the 25 test subjects continued to voluntarily undergoing treatment under the Potaba protocol described herein. While some patients responded to the Potaba treatment protocol with significant alleviation of chronic pain in short periods of time and others after longer periods of time, the average period of time for a subject to undergo Potaba therapy and to report a noticeable and significant reduction in chronic pain was four months. Therefore, the preferred period of time for a subject to follow the treatment plan of the present invention is four months. It is, however, within the scope of the present invention for some subjects to follow the Potaba protocol of the present invention for very short periods of time, which can be repeated on an as-needed basis or to continue the Potaba protocol without interruption for a prolonged or continuing time period so as to avoid a recurrence of the fibrotic condition associated with pelvic pain. It is also within the concept of the present invention to administer the Potaba protocol to a subject and then to discontinue such administration, if effective, after only one dose. In contrast, some patients may suffer such chronic pain that discontinuance of the Potaba protocol immediately results in a recurrence of that pain and, in such cases, a prolonged period of time for following the protocol may be required. Such prolonged administration or maintenance dosage protocol for the administration of Potaba to a subject can be open-ended and limited in time only by the medical judgment of the attending physician and such protocol is still considered within the concept of the present invention.

The remainder of the 212 women under clinical examination who were not part of the 25 subjects undergoing the Potaba protocol test, but who still required treatment for endometriosis and chronic abdominal pain received conventional treatments and exhibited conventionally inadequate results.

The Potaba protocol test clearly showed that the administration of Potaba, as compared to known and conventional treatments, is highly effective in the treatment of subjects suffering from pain due to fibrosis, adhesions resulting from fibrosis, and more particularly, chronic pelvic pain due to endometriosis. The test protocol reaffirmed that oral administration of Potaba is non-toxic, it provides a low risk treatment compared to conventional treatments, it can be used in treatment protocols that usually are under long term conditions, there is a low incidence of drug interactions, it is highly soluble in water, and it is readily absorbed in the gastrointestinal tract.

Potaba has been used for dermatological, urological, and autoimmune diseases with varying degrees of success. In such previous uses of Potaba, it has been shown to be well-tolerated by patients, albeit with some known side effects of oral administration, such as, for example: anorexia, nausea, fever, and rash. While such side effects may occur in the method of the present invention, the surprisingly low dosage required to achieve effective relief of chronic pain due to endometriosis serves to minimize the occurrence and discomfort associated with those side effects.

In the present invention it was discovered and clinically shown that the oral administration of aminobenzoate potassium (Potaba) is a safe, effective treatment to alleviate the pain caused by the adhesions that result from a fibrotic condition such as found with endometriosis.

While the present invention has been described with reference to specific embodiments and the exemplary method of treating chronic pelvic pain associated with endometriosis, it is understood by those skilled in the art that the method disclosed herein is applicable to the treatment of pain associated with fibrotic conditions and resultant adhesions regardless of etiology. Accordingly, a variety of changes may be made and the substitution of equivalents may be made without departing from the true spirit and scope of the present invention. Any modifications that may be made to the exemplary method of the present invention may be made to adapt a particular situation or a particular selected cause of the fibrotic condition to the inclusive concept of the present invention.

1. A method of treating a fibrotic condition with associated chronic pain in a subject in need of such treatment, the method comprising:
providing a composition containing an effective amount of aminobenzoate potassium;
administering said composition to said subject in a therapeutically effective dose over a period of time sufficient to relieve said chronic pain.

2. The method of claim 1, wherein fibrous adhesions are associated with said fibrotic condition.

3. The method of claim 2, wherein said condition is selected from the group consisting of cirrhosis of the liver, atherosclerosis, dermatomyositis, progressive systemic sclerosis, pulmonary fibrosis, connective tissue diseases, adhesions and endometriosis.

4. The method of claim 2, wherein said fibrotic condition is endometriosis.

5. The method of claim 1, wherein said therapeutically effective amount is about 2 grams to about 30 grams per day.

6. The method of claim 1, wherein said therapeutically effective amount is about 10 grams to about 15 grams per day.

7. The method of claim 1, wherein said period of time is about 2 to 6 months.

8. The method of claim 1, wherein said period of time is about 3 to 5 months.

9. The method of claim 1, wherein said period of time is prolonged so as to provide an open-ended maintenance dosage protocol.

10. The method of claim 1, wherein said period of time encompasses the administration of only one dose of said composition.

11. The method of claim 1, wherein said dose is provided in a plurality of daily doses over said period of time.

12. The method of claim 11, wherein each daily dose of said daily doses can be subdivided so as to administer said composition in more than one dose per day.

13. The method of claim 1, wherein said composition is administered by at least one method of administration selected from the group consisting of oral, rectal, transdermal delivery, direct peritoneal application, peritoneal infusion, time-release pellet implant in the body tissues, tissue injection, vaginal insertion, and local direct application on adhesions.

14. The method of claim 13, wherein said oral administration comprises the administration of a time-release capsule.

15. The method of claim 13, wherein said tissue injection is by intramuscular, intravenous, intraperitoneal, or subcutaneous injection.

16. The method of claim 13, wherein said transdermal delivery of said composition is with or without a carrier component, said carrier component being water, chemical, and/or patch.

17. The method of claim 17, wherein said carrier is a lotion, cream, ointment, or salve.

18. The method of claim 13, wherein said direct peritoneal insertion or said peritoneal infusion is accomplished through use of a laproscope, trocar, cannula, catheter, or like device.

19. A method of a subject in need of treatment for pain, the method comprising:
providing a composition containing an effective amount of aminobenzoate potassium;
administering said composition to said subject in a therapeutically effective dose over a period of time sufficient to relieve said pain.

20. The method of claim 19, wherein said therapeutically effective amount is about 2 grams to about 30 grams per day.

21. The method of claim 19, wherein said therapeutically effective amount is about 10 grams to about 15 grams per day.

22. The method of claim 19, wherein said period of time is about 2 to 6 months.

23. The method of claim 19, wherein said period of time is about 3 to 5 months.

24. The method of claim 19, wherein said period of time is prolonged so as to provide an open-ended maintenance dosage protocol.

25. The method of claim 19, wherein said period of time encompasses the administration of only one dose of said composition.

26. The method of claim 19, wherein said dose is provided in a plurality of daily doses over said period of time.

27. The method of claim 26, wherein each daily dose of said daily doses can be subdivided so as to administer said composition in more than one dose per day.

28. The method of claim 19, wherein said composition is administered by at least one method of administration selected from the group consisting of oral, rectal, transdermal delivery, direct application, infusion, time-release pellet implant in the body tissues, tissue injection, vaginal insertion, and local direct application.

29. The method of claim 28, wherein said oral administration comprises the administration of a time-release capsule.

30. The method of claim 28, wherein said transdermal delivery of said composition is with or without a carrier component, said carrier component being water, chemical, and/or patch.

31. The method of claim 30, wherein said carrier is a lotion, cream, ointment, or salve.

32. The method of claim 28, wherein said direct peritoneal insertion or said peritoneal infusion is accomplished through use of a laproscope, trocar, cannula, catheter, or like device.
